(19) United States
(12) Patent Application Publication
(10) Pub. No.: US 2026/0123906 A1
(43) Pub. Date: May 7, 2026

(54) Ultrasound Imaging System

(71) Applicant: Bard Access Systems, Inc., Salt Lake City, UT (US)

(72) Inventors: Steffan Sowards, Salt Lake City, UT (US); William Robert McLaughlin, Bountiful, UT (US); Anthony K. Misener, Bountiful, UT (US)

(21) Appl. No.: 19/440,091

(22) Filed: Jan. 5, 2026

Related U.S. Application Data
(62) Division of application No. 17/684,180, filed on Mar. 1, 2022, now Pat. No. 12,514,532.

(51) Int. Cl. A61B 8/06; A61B 8/00; A61B 8/08

(52) U.S. Cl. CPC A61B 8/06; A61B 8/0891; A61B 8/4245; A61B 8/4477; A61B 8/4488; A61B 8/463; A61B 8/469; A61B 8/488

(57) ABSTRACT

An ultrasound imaging system configured to assess the impact of placement of a vascular access device on fluid flow through a target vessel. The ultrasound imaging system includes an ultrasound probe having an ultrasound array configured to capture one or more ultrasound images of the target vessel and a Doppler array configured to detect the fluid flow through a region of interest of the target vessel. The ultrasound imaging system further includes a console in communication with each of the ultrasound array and the Doppler array, the console configured to determine the region of interest of the target vessel.

### PRIORITY

This application is a division of U.S. patent application Ser. No. 17/684,180, filed Mar. 1, 2022, now U.S. Pat. No. 12,514,532, which is incorporated by reference in its entirety into this application.

### BACKGROUND

Current methods for placing a vascular access device include placing a vascular access device occupying no more than 45% of a target vessel. Ultrasound imaging systems are used to detect vascular access device occupancy within the target vessel. However, these methods do not ensure that blood flow through the vessel is not compromised once the vascular access device is placed within the vessel. It would be beneficial to the clinician and the patient to have an ultrasound imaging system that may be used to image the target vessel, detect occupancy of the vascular access device within the target vessel, and assess blood flow through the target vessel after the vascular access device has been placed. Disclosed herein is an ultrasound imaging system and method of use that address the foregoing.

### SUMMARY

Disclosed herein is an ultrasound imaging system configured to assess the impact of placement of a vascular access device on fluid flow through a target vessel. The ultrasound imaging system includes an ultrasound probe having an ultrasound array configured to capture one or more ultrasound images of the target vessel and a Doppler array configured to detect the fluid flow through a region of interest of the target vessel. The ultrasound imaging system further includes a console in communication with each of the ultrasound array and the Doppler array, the console configured to determine the region of interest of the target vessel.

In some embodiments, the console is configured to determine the total cross sectional area of the target vessel using one or more ultrasound images.

In some embodiments, the console assesses the impact of the vascular access device on fluid flow through the target vessel by comparing a first flow rate value of the region of interest before placement of the vascular access device with a second flow rate value of the region of interest after placement of the vascular access device.

In some embodiments, the first flow rate value and the second flow rate value are detected distal an insertion site of the vascular access device.

In some embodiments, the impact of the vascular access device on fluid flow through the target vessel is expressed as a percent of the total cross sectional area of the target vessel.

In some embodiments, the console includes one or more processors, an energy source, non-transitory computer readable medium and a plurality of logic modules that when activated by the process are configured to perform operations including tracking the ultrasound probe within the target area, receiving the one or more ultrasound images from the ultrasound array, determining the target vessel and the cross sectional area of the target vessel within the one or more ultrasound images, receiving flow rate data from the Doppler array, determining the flow rate through the region of interest and generating a flow rate value, determining the region of interest, comparing a first flow rate value with a second flow rate value, and generating and overlaying a flow rate value icon on the one or more ultrasound images.

In some embodiments, the user selects the region of interest on a display in communication with the console.

In some embodiments, the console is configured to detect vascular access device occupancy within the target vessel, generate a vascular access device occupancy value, and compare the vascular access device value with the impact of the vascular access device on fluid flow through the target vessel as expressed as the percent of the total cross sectional area of the target vessel.

Also disclosed herein is a method of assessing fluid flow through a target vessel before and after placement of a vascular access device including imaging the target vessel within a target area using an ultrasound imaging system, identifying a region of interest along the target vessel, assessing fluid flow through the region of interest, placing a vascular access device within the target vessel, imaging the target vessel after placement of the vascular access device within the target vessel, and assessing fluid flow through the region of interest after placement of the vascular access device within the target vessel.

In some embodiments, imaging the target vessel within the target area using the ultrasound imaging system includes imaging the target vessel with an ultrasound probe having an ultrasound array and a Doppler array, each in communication with a console.

In some embodiments, imaging the target vessel within the target area using the ultrasound imaging system includes imaging the target vessel at a first location within the target area.

In some embodiments, identifying the region of interest along the target vessel includes the console automatically identifying the region of interest.

In some embodiments, identifying the region of interest along the target vessel includes a user identifying the region of interest on a display in communication with the console.

In some embodiments, assessing fluid flow through the region of interest includes the detecting a first flow rate value through the region of interest.

In some embodiments, detecting the first flow rate through the region of interest includes detecting a first flow rate value at the first location within the target area.

In some embodiments, detecting the first flow rate value at the first location within the target area includes the first location being distal an insertion site.

In some embodiments, imaging the target vessel after placement of the vascular access device within the target vessel includes imaging the target vessel within the target area.

In some embodiments, imaging the target vessel within the target area includes imaging the target vessel at the first location distal the insertion site.

In some embodiments, assessing fluid flow through the region of interest after placement of the vascular access device within the target vessel includes detecting a second flow rate value through the region of interest after the vascular access device has been placed within the target vessel.

In some embodiments, detecting the second flow rate value includes detecting the second flow rate value at the first location distal the insertion site.

In some embodiments, assessing fluid flow through the region of interest after placement of the vascular access device within the target vessel includes comparing the second flow rate value with the first flow rate value to confirm fluid flow through the target vessel has not been compromised by the placement of the vascular access device.

These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of an ultrasound imaging system, in accordance with some embodiments.

FIG. 2 illustrates a perspective view of a display of the ultrasound imaging system, in accordance with some embodiments.

FIGS. 3A-3C illustrate a cross sectional view of an exemplary method of assessing blood flow through a target vessel before and after vascular access device insertion, in accordance with some embodiments.

FIG. 4 illustrates a block diagram of a console of the ultrasound imaging system, in accordance with some embodiments.

FIG. 5 illustrates a flow chart of an exemplary method of placing a vascular access device, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

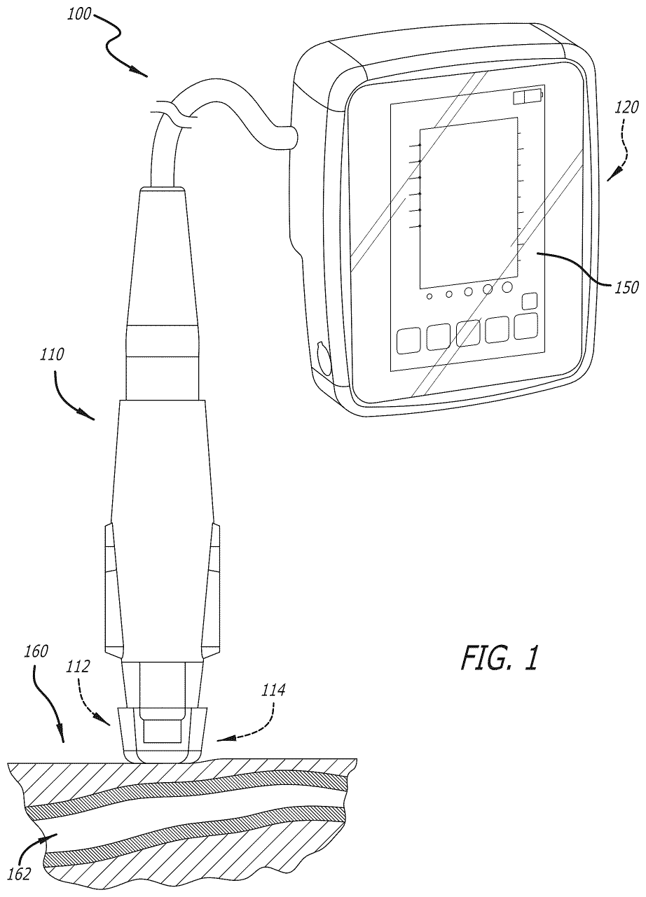

FIG. 1 illustrates a perspective view of an ultrasound imaging system 100, in accordance with some embodiments. In some embodiments, the ultrasound imaging system 100 includes an ultrasound probe 110 in communication with a console 120. In some embodiments, the console 120 may be in communication with a display 150 configured to display one or more ultrasound images. In some embodiments, the ultrasound probe 110 includes an ultrasound array 112 and a Doppler array 114. In some embodiments, the ultrasound array 112 may be configured to capture one or more images of a target vessel 162 within a target area 160 and communicate the one or more images to the console 120. In some embodiments, the Doppler array 114 may be configured to detect fluid flow including flow rate through the target vessel 162 and communicate the detected fluid flow data to the console 120. In some embodiments, the ultrasound array 112 may be configured to simultaneously operated at the same time as the Doppler array 114, or the ultrasound array 112 may be continuously running and the Doppler array 114 may be configured to be selectably switched on, depending on a user's imaging needs.

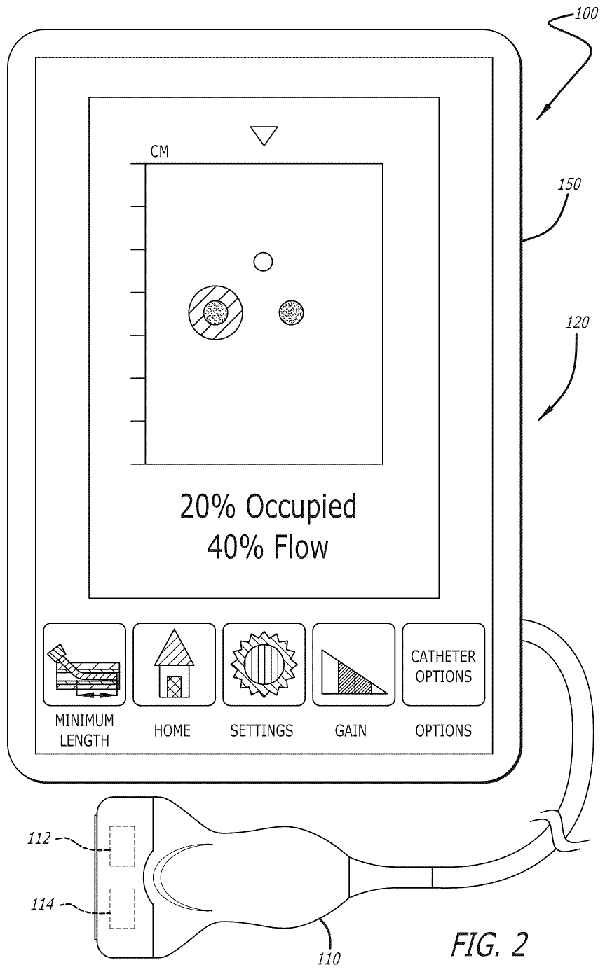

FIG. 2 illustrates a perspective view of the display 150 of the ultrasound imaging system 100, in accordance with some embodiments. In some embodiments, the console 120 may be coupled to the display 150. In some embodiments, the display 150 may include a touch screen display. In some embodiments, the display 150 may be configured to show the one or more ultrasound images captured from the ultrasound array 112 and may be configured to overlay the data received from the Doppler array 114 onto the one or more ultrasound images. In some embodiments, a user may select, on the display 150, a region of interest of the target vessel 162. In some embodiments, the region of interest may include the target vessel 162 or a portion of the target vessel 162. In some embodiments, when the region of interest is selected, the display 150 may display ultrasound images from the region of interest including fluid flow data from the Doppler array 114, as will be described in more detail herein. In some embodiments, the display 150 may be configured to display a variety of icons including a target vessel occupancy icon, a fluid flow rate icon, a region of interest icon, or the like. In some embodiments, the Doppler array 114 may be able to be turned on or turned off including from the display 150 or from the ultrasound probe 110. In some embodiments, the variety of icons may include different shapes, different colors, or different sizes. In some embodiments, the variety of icons may change in response to data received by the console 120. For example, in some embodiments, the variety of icons may change in response to an increase or decrease in fluid flow rate.

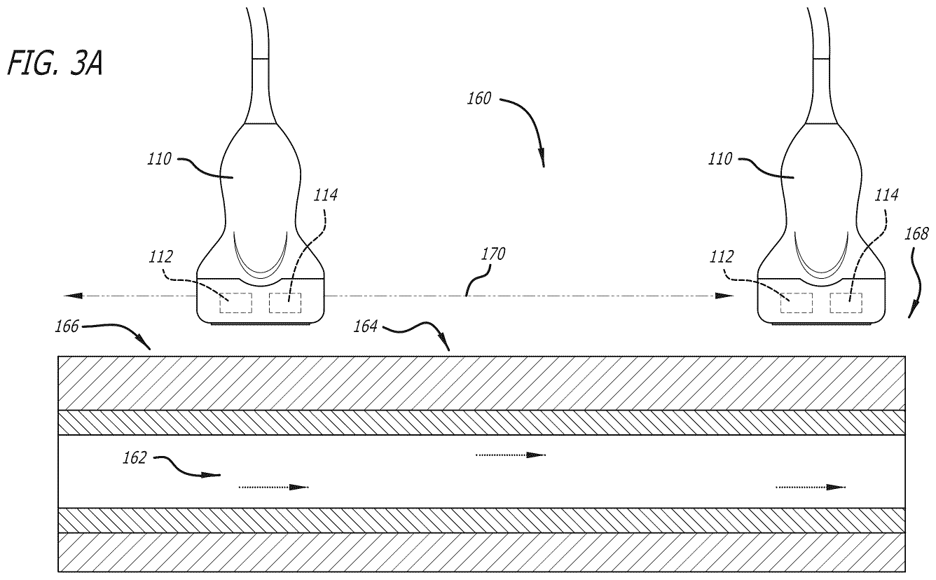

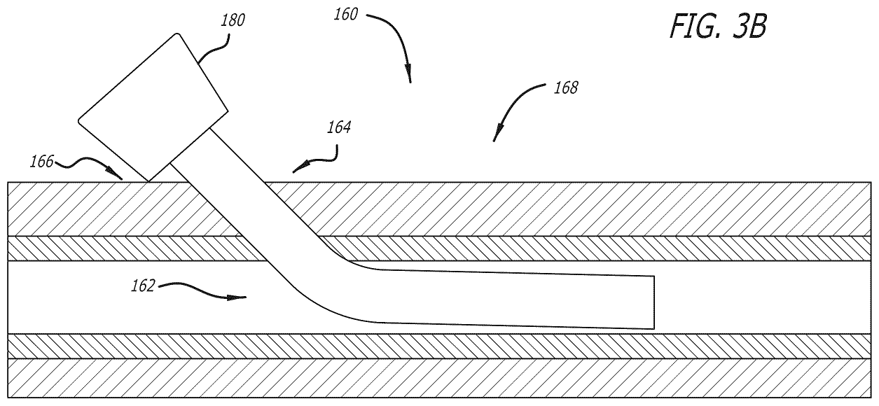

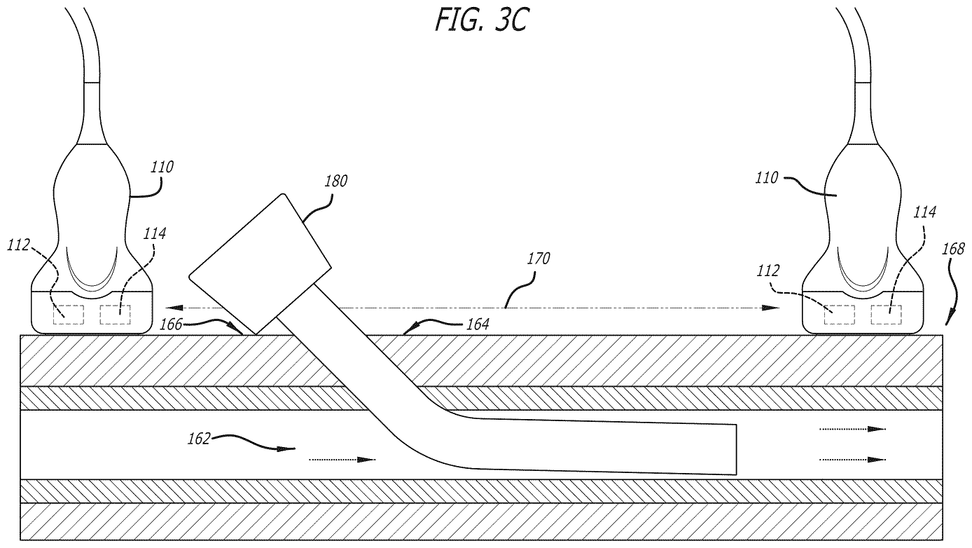

FIGS. 3A-3C illustrate a cross sectional view of an exemplary method of assessing fluid flow through the target vessel 162 before and after insertion of the vascular access device 180, in accordance with some embodiments. In some embodiments, the ultrasound probe 110 may be used to image the target vessel 162 within the target area 160. The ultrasound probe 110 may be brought to a skin surface within the target area 160. The ultrasound array 112 may be used to capture one or more images of the target vessel 162. The Doppler array 114 may be used to assess the blood flow through the target vessel 162. In some embodiments, the ultrasound probe 110 may be configured to capture one or more images of the target vessel 162 along an entire first length 170, wherein the first length 170 includes an insertion site 164. In some embodiments, the ultrasound probe 110 may be configured to image the target vessel 162 proximal the insertion site 164 and distal the insertion site 164. In some embodiments, the first length 170 may include adjacent or proximal to the insertion site 164, including a first side 166 of the insertion site 164 and a second side 168 of the insertion site 164.

As illustrated in FIG. 3B, once the target vessel 162 has been imaged, the vascular access device 180 may be placed within the target vessel 162 at the insertion site 164. In some embodiments, a portion of the vascular access device 180 may reside within the target vessel 162 and impede a portion of flow through the target vessel 162. With the vascular access device 180 placed within the target vessel 162, the target vessel 162 may be imaged along the first length 170 by the ultrasound probe 110, including proximal the insertion site 164 and distal the insertion site 164. The Doppler array 114 may be used to detect fluid flow through the target vessel 162 including fluid flow proximal the insertion site 164 on the first side 166 and fluid flow distal the insertion site 164 on the second side 168. In some embodiments, the console 120 may be configured to assess the fluid flow through the target vessel 162 to determine the impact the placement of the vascular access device 180 within the target vessel 162 has on fluid flow through the target vessel 162 (e.g., if fluid flow is significantly disrupted with the vascular access device 180 placed therein). In some embodiments, the console 120 may be configured to determine the cross sectional area of the target vessel 162 and display one or more flow rate values of the flow rate through the region of interest including around the vascular access device 180 as a percent of the total cross sectional area of the target vessel 162.

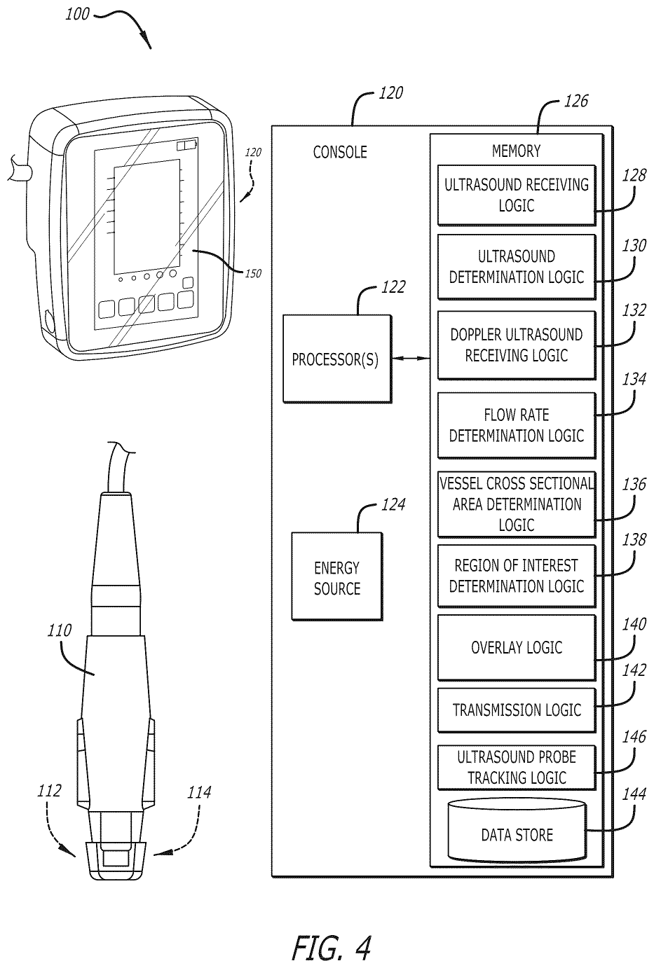

FIG. 4 illustrates a block diagram of the console 120, in accordance with some embodiments. In some embodiments, the console 120 may be in communication with each of the ultrasound probe 110 and the display 150. In some embodiments, the console 120 may be coupled to the ultrasound probe 110, wired to the ultrasound probe 110, may be wired to the display 150, coupled to the display 150 or may be in wireless communication with each of the ultrasound probe 110 and the display 150, or a combination thereof. In some embodiments, the console 120 may include one or more processors 122, an energy source 124, non-transitory computer readable medium (“memory”) 126, and a plurality of logic modules. In some embodiments, the plurality of logic modules may include one or more of: ultrasound receiving logic 128, ultrasound determination logic 130, Doppler array receiving logic 132, flow rate determination logic 134, vessel cross sectional area determination logic 136, region of interest determination logic 138, overlay logic 140, a transmission logic 142, an ultrasound probe tracking logic 146, and a data store 144.

In some embodiments, the ultrasound receiving logic 128 may be configured to receive the one or more ultrasound images captured from the ultrasound array 112 on the ultrasound probe 110. In some embodiments, the ultrasound receiving logic 128 may be configured to generate a time of day value that may be associated with each received ultrasound image. In some embodiments, the ultrasound receiving logic 128 may be configured to receive the coordinates of the ultrasound probe 110 within the target area 160 when the ultrasound array 112 acquired the one or more ultrasound images. In some embodiments, the ultrasound determination logic 130 may be configured to determine and define the target vessel 162 within the one or more ultrasound images. In some embodiments, the Doppler array receiving logic 132 may be configured to receive flow rate data (e.g., Doppler ultrasound values) from the Doppler array 114 including the flow rate through the target vessel 162 and the coordinates of the ultrasound probe 110 within the target area 160 when the Doppler array 114 acquired the flow rate data.

In some embodiments, the flow rate determination logic 134 may be configured to use the flow rate data acquired by the Doppler array 114 at a first time point to determine the flow rate through the region of interest and generate one or more flow rate values corresponding to the flow rate through the region of interest. In some embodiments, the flow rate determination logic 134 may be configured to generate an average flow rate value for the flow rate along the first length 170 or may be configured to generate a flow rate value for each coordinate within the target area 160 where the Doppler array 114 acquired flow rate data. In some embodiments, the flow rate determination logic 134 may compare the flow rate data (e.g., flow rate values) acquired from pre-insertion of the vascular access device 180 at a first time point to flow rate data (e.g., flow rate values) post-insertion of the vascular access device 180 at a second time point. For example, the flow rate determination logic 134 may be configured to generate a first flow rate value from flow rate data received at a first location distal the insertion site 164 pre-insertion of the vascular access device 180 and then generate a second flow rate value from flow rate data received at the first location distal the insertion site 164 post-insertion of the vascular access device 180.

In some embodiments, the flow rate determination logic 134 may compare the flow rate data including flow rate values from the same coordinates within the target area 160. In some embodiments, the flow rate determination logic 134 may be configured to compare historical flow rate data of the region of interest to presently acquired flow rate data. For example, the flow rate determination logic 134 may compare historical flow rate data of the region of interest to presently acquired flow rate data pre-insertion of the vascular access device 180 and post-insertion of the vascular access device 180.

In some embodiments, the vessel cross sectional area determination logic 136 may be configured to determine the cross sectional area of the target vessel 162 from one or more ultrasound images imaged using the ultrasound array 112. In some embodiments, the vessel cross sectional area determination logic 136 may be configured to generate a total cross sectional area value. In some embodiments, the vessel cross sectional area determination logic 136 determines the outer region of the target vessel 162 which provides the total percentage possible in terms of fluid flow through the cross section of the target vessel 162. In some embodiments, the vessel cross sectional area determination logic 136 may be configured to compare the flow rate values to the total cross sectional area value to determine what percentage of the total cross sectional area of the target vessel 162 is having fluid flow therethrough.

In some embodiments, the vessel cross sectional area determination logic 136 may be configured to express the fluid flow rate through the region of interest as a percentage of the total cross sectional area of the target vessel 162 or as a flow rate ratio. In some embodiments, the vessel cross sectional area determination logic 136 may be configured to compare the flow rate values after placement of the vascular access device 180 with flow rate values before the placement of the vascular access device 180. In some embodiments, the vessel cross sectional area determination logic 136 may be configured to determine that fluid flow through the target vessel 162 has been significantly compromised by comparing flow rate values after placement of the vascular access device 180 with a flow rate value threshold to determine if fluid flow through the target vessel 162 has been compromised. In some embodiments, the flow rate value threshold may be user selected or automatically determined using historical flow rate values including flow rate values before placement of the vascular access device 180.

In some embodiments, the region of interest determination logic 138 may be configured to automatically determine the region of interest. In some embodiments, the region of interest determination logic 138 may be configured to use detected anatomical targets in proximity to the target vessel 162 in order to determine the region of interest. In some embodiments, the region of interest determination logic 138 may be configured to be determined using user input (e.g., the user may select the region of interest on the display 150 or the like). In some embodiments, the region of interest determination logic 138 may be configured to be determined using historical data (e.g., previous ultrasound images) to determine the region of interest. In some embodiments, the region of interest determination logic 138 may be configured to be determined using a fixed distance from the insertion site 164.

In some embodiments, the overlay logic 140 may be configured to generate a flow rate data icon and overlay the flow rate data received from the Doppler array 114 or the flow rate data icon on the one or more ultrasound images received from the ultrasound array 112. In some embodiments, the flow rate data icon may have various shapes, sizes, or colors, wherein each shape, size, or color may correspond to a specific flow rate value (e.g., a heat map). In some embodiments, overlay logic 140 may be configured to match the coordinates of the ultrasound images and the coordinates of the flow rate data to overlay the flow rate data or flow rate data icon over the proper location of the region of interest in the one or more ultrasound images.

In some embodiments, the transmission logic 142 may be configured to transmit the one or more ultrasound images to the display 150. In some embodiments, the transmission logic 142 may be configured to transmit the overlay of the Doppler ultrasound data on the one or more ultrasound images to the display 150. In some embodiments, the transmission logic 142 may be configured to transmit the fluid flow rate through the region of interest as a percentage of the total cross sectional area of the target vessel 162 to the display 150.

In some embodiments, the ultrasound probe tracking logic 146 may be configured to track the coordinates of the ultrasound probe 110 within the target area 160. In some embodiments, the ultrasound probe tracking logic 146 may be configured to always be tracking the coordinates of the ultrasound probe 110 within the target area 160 or the ultrasound probe tracking logic 146 may be configured to track the coordinates of the ultrasound probe 110 only when the ultrasound probe tracking logic 146 records the ultrasound array 112 or the Doppler array 114 is activated.

In some embodiments, the data store 144 may be configured to store historical flow rate data from the target vessel 162. In some embodiments, the data store 144 may be configured to store each of the one or more ultrasound images acquired from the ultrasound array 112 and the corresponding data from the Doppler array 114. In some embodiments, the data store 144 may be configured to store the ultrasound images and Doppler ultrasound data as {(ultrasound image coordinates), (time of day value), (Doppler ultrasound coordinates), (flow rate value)} or the like.

In some embodiments, the console 120 may be configured to measure, detect, or predict target vessel occupancy of the vascular access device 180 including an assessment of the vascular access device 180 to target vessel 162 ratio. In some embodiments, the console 120 may be configured to generate a vascular access device occupancy value or the vascular access device occupancy ratio (e.g., a ratio of the cross sectional area of the vascular access device 180 to the cross sectional area of the target vessel 162). In some embodiments, the console 120 may be configured to compare the detected or predicted vessel occupancy, or vascular access device occupancy ratio with the flow rate through the region of interest expressed as a percent of the total cross sectional area of the target vessel 162 to confirm proper vessel occupancy.

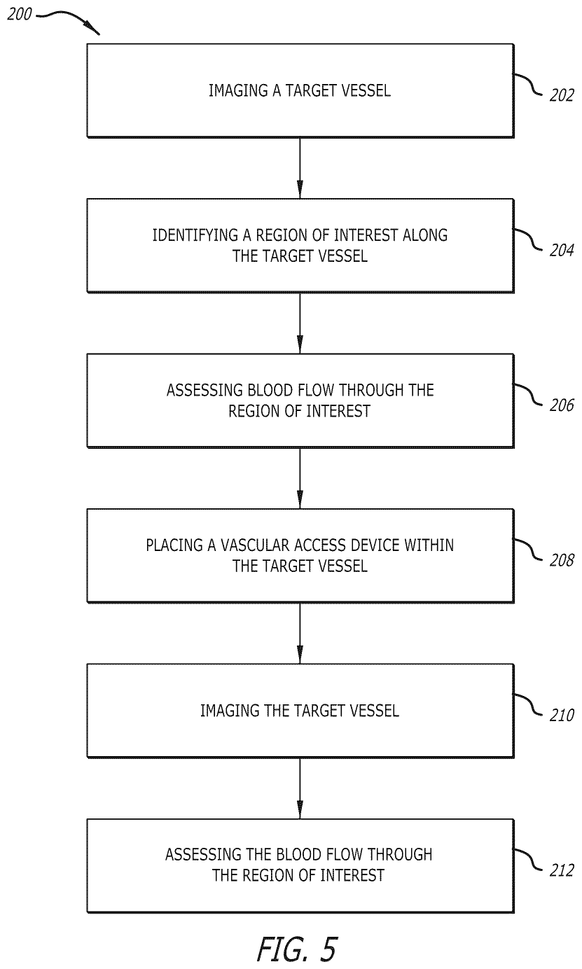

FIG. 5 illustrates a flow chart of an exemplary method 200 of placing a vascular access device 180 within a target vessel 162, in accordance with some embodiments. In some embodiments, the method 200 includes imaging a target vessel 162 in a target area 160 using an ultrasound imaging system 100 (block 202). In some embodiments, imaging the target vessel 162 within the target area 160 using the ultrasound imaging system 100 includes imaging the target vessel 162 using the ultrasound probe 110 having the ultrasound array 112 and the Doppler array 114, each in communication with the console 120. In some embodiments, imaging the target vessel 162 includes the console 120 transmitting the one or more ultrasound images to the display 150. In some embodiments, imaging the target vessel 162 in a target area 160 using the ultrasound imaging system 100 includes imaging the target vessel 162 at a first location within the target area 160. In some embodiments, imaging the target vessel 162 in the target area 160 includes using the ultrasound imaging system 100 to image the target vessel 162 along the first length 170.

In some embodiments, the method 200 includes identifying a region of interest along the target vessel 162 (block 204). In some embodiments, identifying the region of interest along the target vessel 162 includes the user identifying the region of interest along the target vessel 162. In some embodiments, identifying the region of interest along the target vessel 162 includes the user selecting the region of interest on the display 150. In some embodiments, identifying the region of interest along the target vessel 162 includes the console 120 automatically identifying the region of interest along the target vessel 162. In some embodiments, the console 120 automatically identifying the region of interest along the target vessel 162 includes the console 120 using anatomical targets within the target area 160 to identify the region of interest along the target vessel 162. In some embodiments, identifying the region of interest includes the console 120 selecting the coordinates of the region of interest within the target area 160.

In some embodiments, the method 200 includes assessing fluid flow through the region of interest (block 206). In some embodiments, assessing fluid flow through the region of interest includes the console 120 using data received from the Doppler array to determine the fluid flow through the target vessel 162. In some embodiments, assessing fluid flow through the region of interest includes detecting a first flow rate value through the region of interest. In some embodiments, detecting the first flow rate value through the region of interest includes detecting the first flow rate value at the first location within the target area 160. In some embodiments, the first location may be distal the insertion site 164 In some embodiments, assessing fluid flow through the region of interest includes the console 120 transmitting the fluid flow rate to the display 150 as a percent of the total cross sectional area of the target vessel 162. In some embodiments, assessing fluid flow through the region of interest occurs simultaneously with imaging the target vessel 162.

In some embodiments, the method 200 includes placing a vascular access device 180 within the target vessel 162 (block 208). In some embodiments, placing the vascular access device 180 within the target vessel 162 includes placing the vascular access device 180 into the target vessel 162 through an insertion site 164 within the target area 160. In some embodiments, placing the vascular access device 180 within the target vessel 162 includes using the Seldinger technique to place the vascular access device 180 within the target vessel 162. In some embodiments, placing the vascular access device 180 within the target vessel 162 includes slidably inserting the vascular access device 180 into the target vessel 162.

In some embodiments, the method 200 includes imaging the target vessel 162 after placement of the vascular access device 180 within the target vessel 162 (block 210). In some embodiments, imaging the target vessel 162 after placement of the vascular access device 180 within the target vessel 162 includes using the ultrasound probe 110 having the ultrasound array 112 and the Doppler array 114 to image the target vessel 162. In some embodiments, imaging the target vessel 162 includes imaging the target vessel 162 along the first length 170 within the target area 160. In some embodiments, imaging the target vessel 162 includes imaging the target vessel 162 along the first length 170 including the first side 166 of the insertion site 164 and the second side 168 of the insertion site 164. In some embodiments, imaging the target vessel 162 includes using the Doppler array 114 to image the target vessel 162 at the same coordinates as previously captured ultrasound images within the target area 160. In some embodiments, imaging the target vessel 162 after placement of the vascular access device 180 within the target vessel 162 includes imaging the target vessel 162 at the first location distal the insertion site 164. In some embodiments, imaging the target vessel 162 after placement of the vascular access device 180 within the target vessel 162 includes imaging the target vessel 162 at a first side 166 of the insertion site 164 and a second side 168 of the insertion site 164.

In some embodiments, the method 200 includes assessing the fluid flow through the region of interest after placement of the vascular access device 180 within the target vessel 162 (block 212). In some embodiments, assessing the fluid flow through the region of interest after placement of the vascular access device 180 within the target vessel 162 includes the console 120 using the data received from the Doppler array 114 to determine the flow rate through the target vessel 162 including through the region of interest with the vascular access device 180 placed therein. In some embodiments, assessing the fluid flow through the region of interest after placement of the vascular access device 180 within the target vessel 162 includes assessing fluid flow while simultaneously imaging the target vessel 162.

In some embodiments, assessing the fluid flow through the region of interest after placement of the vascular access device 180 within the target vessel 162 includes detecting a second flow rate value through the region of interest after the vascular access device has been placed within the target vessel 162. In some embodiments, detecting the second flow rate value includes detecting the second flow rate value at the first location distal the insertion site 164. In some embodiments, assessing fluid flow through the region of interest after placement of the vascular access device 180 within the target vessel 162 include comparing the second flow rate value with the first flow rate value to confirm fluid flow through the target vessel 162 has not be compromised by the placement of the vascular access device 180 within the target vessel 162. In some embodiments, assessing the fluid flow through the region of interest includes the console 120 communicating the fluid flow rate through the target vessel 162 after placement of the vascular access device 180 to the display 150 as a percent of the total cross sectional area of the target vessel 162.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An ultrasound imaging system configured to assess the impact of placement of a vascular access device on fluid flow through a target vessel, comprising:
an ultrasound probe, comprising:
an ultrasound array configured to capture one or more ultrasound images of the target vessel; and
a Doppler array configured to detect the fluid flow through a region of interest of the target vessel; and
a console in communication with each of the ultrasound array and the Doppler array, the console configured to perform operations comprising:
displaying an image of the target vessel within a target area on a display of the console;
automatically selecting a portion of the target vessel as a region of interest along the target vessel via logic operations of the console
using anatomical targets in proximity to the target vessel within the target area;
obtaining first fluid flow rate data from the Doppler array indicating a first fluid flow rate value through the region of interest prior to placement of the vascular access device within the target vessel;
imaging the target vessel after placement of the vascular access device within the target vessel;
obtaining second fluid flow rate data from the doppler array indicating a second fluid flow rate value through the region of interest after placement of the vascular access device within the target vessel; and
detecting that fluid flow through the target vessel has been compromised based on results of comparing the second fluid flow rate value with a flow rate value threshold.

2. The ultrasound imaging system according to claim 1, wherein the console is configured to determine a total cross sectional area of the target vessel using one or more ultrasound images.

3. The ultrasound imaging system according to claim 2, wherein the impact of the vascular access device on fluid flow through the target vessel is expressed as a percent of the total cross sectional area of the target vessel.

4. The ultrasound imaging system according to claim 1, wherein the first flow rate value and the second flow rate value are detected distal an insertion site of the vascular access device.

5. The ultrasound imaging system according to claim 1, wherein imaging the target vessel includes imaging the target vessel at a first location within the target area, and wherein the first fluid flow rate data from the Doppler array indicates the first fluid flow rate value at the first location within the target area.

6. The ultrasound imaging system according to claim 5, wherein the first location is distal an insertion site of the vascular access device.

7. The ultrasound imaging system according to claim 6, wherein the second fluid flow rate data from the Doppler array indicates the second fluid flow rate value at the first location distal the insertion site.

8. The ultrasound imaging system according to claim 1, wherein automatically selecting a portion of the target vessel as the region of interest includes using historical data from previously acquired ultrasound images of the target area to identify the region of interest.

9. The ultrasound imaging system according to claim 1, wherein automatically selecting a portion of the target vessel as the region of interest includes using a fixed distance from an insertion site to identify the region of interest.

10. The ultrasound imaging system according to claim 1, wherein the console is configured to perform further operations comprising:
generating a flow rate data icon overlay including the first fluid flow rate data, and
matching coordinates of the image of the target vessel with coordinates of the flow rate data icon overlay to locate the flow rate data icon overlay at the region of interest.

11. The ultrasound imaging system according to claim 1, wherein the console is configured to perform further operations comprising:
determining a total cross-sectional area value of the target vessel from the image of the target vessel, and
determining a vascular access device occupancy percentage based on the total cross-sectional area value and a cross-sectional area value of the vascular access device.
